SEQUENCE LISTING (4 sequences)

SEQ ID NO: 1 - protein, 40 aa, Salmonella enterica
MQRGIVWVVDDDSSIRWVLERALAGAGLTCTTFENGNEVL

SEQ ID NO: 2 - protein, 40 aa, Artificial Sequence
  de novo designed mini-protein
TQTQEFDNEEEARKAEKELRKENRRVTVTQENGRWRVTWD

SEQ ID NO: 3 - protein, 44 aa, Artificial Sequence
  de novo designed protein
APCEDLKERLKKLGMSEECRQRLEKMCKEGTSEDAERMARNCES

SEQ ID NO: 4 - protein, 33 aa, Saccharomyces cerevisiae
RMKQLEDKVEELLSKNYHLENEVARLKKLVGER

(19) United States
(12) Patent Application Publication
(10) Pub. No.: US 2022/0415432 A1
(43) Pub. Date: Dec. 29, 2022

(54) Protein Structure Prediction

(71) Applicant: Flagship Pioneering Innovations VI, LLC, Cambridge, MA (US)

(72) Inventor: Gevorg Grigoryan, Arlington, MA (US)

(21) Appl. No.: 17/756,211

(22) PCT Filed: Nov. 20, 2020
(86) PCT No.: PCT/US2020/061526

§ 371 Date: May 19, 2022

Related U.S. Application Data
(60) Provisional application No. 62/938,021, filed on Nov. 20, 2019.

(51) Int. Cl. G16B 15/20; G16B 40/20; G06N 5/02

(52) U.S. Cl. CPC G16B 15/20; G16B 40/20; G06N 5/022

(57) ABSTRACT

The disclosure provides, inter alia, methods of determining the three-dimensional structure of a polypeptide, given a subject protein sequence, e.g., a primary amino acid sequence. The methods can efficiently determine structures, including those of de novo proteins for example, those without known homologues with pre-determined structures.

### RELATED APPLICATION

This application claims the benefit of U.S. Provisional Application No. 62/938,021, filed on Nov. 20, 2019. The entire teachings of the above application are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:

File name: “57081030001_SEQUENCE_LISTING.txt”; created Nov. 19, 2020, 2 KB in size.

### BACKGROUND

It has long been desirable to predict the 3-dimensional (3D) folded structure of a protein from its primary amino-acid sequence. While many methods towards this task have been developed over the years, the problem remains largely unsolved. Accordingly, a need exists for methods, and corresponding systems for implementing the methods, for predicting protein 3D structures, preferably which methods can predict 3D structure for de novo proteins.

### SUMMARY

The disclosure provides, inter alia, methods, and corresponding media and systems for implementing the methods, for predicting protein 3D structures, which methods can advantageously predict 3D structure for de novo proteins and which, in certain embodiments, can advantageously be quickly implemented, e.g., on a personal computer.

In an embodiment, a method of predicting the structure of a subject protein includes accepting the primary amino acid sequence of the subject protein and accessing a library of tertiary motifs. The method further includes providing, to a structural sampler, a topology dataset comprising: a) a set of probable self-tertiary motifs from a library of tertiary motifs for a subject protein and b) a set of probable pair tertiary motifs from the library of tertiary motifs for the subject protein. The method further includes sampling, by the structural sampler, at least one configuration of the subject protein according to the topology dataset. The method further includes calculating, by the structural sampler, a scoring function incorporating a distance between a configuration and the set of probable tertiary motifs. The method further includes generating, by the structural sampler, a predicted structure representing a local minimum according to the scoring function.

In an embodiment, a method of predicting the structure of a subject protein includes initializing a structural sampler with a topology dataset comprising: a) a set of probable self-tertiary motifs from a library of tertiary motifs for a subject protein and b) a set of probable pair tertiary motifs from the library of tertiary motifs for the subject protein. The method further includes sampling, by the structural sampler, at least one configuration of the subject protein according to the topology dataset. The method further includes calculating, by the structural sampler, a scoring function incorporating a distance between a configuration and the set of probable tertiary motifs. The method further includes generating, by the structural sampler, a predicted structure representing a local minimum according to the scoring function.

In an embodiment, a method of predicting the structure of a subject protein includes initializing a structural sampler with a topology dataset. The topology dataset includes a) a set of probable self-tertiary motifs from a library of tertiary motifs for a subject protein and b) a set of probable pair tertiary motifs from the library of tertiary motifs for the subject protein. The method further includes sampling, by the structural sampler, at least one configuration of the subject protein according to the topology dataset. The method further includes calculating, by the structural sampler, a scoring function incorporating a distance between a configuration and the set of probable tertiary motifs. The method further includes generating, by the structural sampler, a predicted structure representing a local minimum according to the scoring function.

In an embodiment, a non-transient computer-readable medium comprising instructions that, upon execution by a microprocessor, causes the microprocessor to perform the method of any one of the preceding embodiments. In a further embodiment, a system can include the non-transient computer-readable medium above and a processor for executing the instructions, optionally wherein the system comprises one or more of a human end-user interface and a means for displaying the predicted structure. In an embodiment, a method of predicting the structure of a subject protein includes providing the above described system with a primary amino acid sequence of the subject protein and obtaining the predicted structure. In an embodiment a method includes steps of any of the above embodiments executed on one or more remote servers (e.g., in the cloud).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a flow diagram of the overall workflow of a structure prediction on a hypothetical sequence of interest (SEQ ID NO: 1) performed in methods provided by the disclosure.

FIGS. 2A-B is a diagram illustrating the process of annotating a hypothetical sequence of interest (SEQ ID NO: 1), given a tertiary motif (TERM) library.

FIG. 3 are folded proteins illustrating the result of applying TERM-based structure prediction to a protein (SEQ ID NO: 2).

FIG. 4 are folded proteins illustrating the result of applying TERM-based structure prediction to a protein (SEQ ID NO: 3).

FIG. 5 are folded proteins illustrating the result of applying TERM-based structure prediction to predict the structure of a parallel coiled coil (SEQ ID NO: 4).

FIG. 6 is a flow diagram illustrating an example embodiment of a method employed by the present disclosure.

FIG. 7 is a block diagram illustrating an example embodiment of the present disclosure for structure prediction on a hypothetical sequence of interest (SEQ ID NO: 1).

FIG. 8 illustrates a computer network or similar digital processing environment in which embodiments of the present invention may be implemented.

FIG. 9 is a diagram of an example internal structure of a computer (e.g., client processor/device or server computers) in the computer system of FIG. 8.

### DETAILED DESCRIPTION

A description of example embodiments follows.

It has long been desirable to predict the 3-dimensional (3D) folded structure of a protein from its primary amino-acid sequence. While many methods towards this task have been developed over the years, the problem remains largely unsolved. Previous methods can be broken down into several broad categories: 1) physics-based approaches, including brute force, 2) statistical approaches, 3) mixed methods (including both statistical and physically motivated components), and more recently 4) machine learning-based methods. To date, the most successful demonstrations of structure prediction come from applications to native proteins (proteins with long evolutionary records), for which deep multiple sequence alignments (MSAs) are available. In these cases, it has been shown by a number of different groups that residue pair co-evolution information, readily extractable from such MSAs, is closely associated to residue-pair proximity in the folded structure. This insight, in turn, can lead to robust structure prediction outcomes in favorable cases. When considering a different regime of structure prediction—predicting the structure of a native protein that lacks deep evolutionary information or a de-novo engineered protein, which was not subject to evolution—advances are much more limited.

Presented herein is an inventive approach to structure prediction that is based on the fundamental observation that the universe of protein structures is degenerate, such that at a local (e.g., in space) level 3D structural geometries of proteins recur and can be categorized. Thus, these recurring, categorized geometries can be rapidly identified in a subject (e.g., query) protein, providing both improved fidelity and speed in structure prediction. Advantageously, the methods provided by the disclosure do not rely on a pre-existing MSA or evolutionary record, and is thus equally applicable to native or engineered proteins.

In a traditional structure prediction/simulation method, the protein geometry (i.e., both local geometry and the global conformation) must emerge from atomistic principles, whether first principles of physics or some derived pseudo-physical scoring function. Current scoring functions operate at the level of residues (i.e., treating each amino acid as a “bead”). These scoring functions provide faster processing, but reduce accuracy to an almost irrecoverable amount.

In contrast, the methods provided by the disclosure consider detailed atomistic patterns without having to discover them from first principles during each simulation. Instead, the methods of the present disclosure employ previously-observed patterns and finds combinations of these patterns that are most suitable for a given sequence. This approach gives tremendous advantages in speed without sacrificing accuracy as in traditional methods.

As an example, consider a simple structure—a short single helix of approximately 20 residues. If a sequence actually folds into this sequence, a traditional structure prediction/simulation method would take a considerable amount of time to discover this fact with an atomistic simulation, because the method waits for the laws of physics to guide a model towards that structure without considering that such a structure is already known to exist in in the protein world. In other words, traditional methods ignore discoveries and knowledge of science, and are as if Linus Pauling had never been born and had never discovered the alpha helix. On the other hand, using the methods provided by the disclosure, it is immediately recognized that a helix is a suitable pattern for the sequence, and can begin considering that configuration immediately or nearly immediately, by realizing that a bunch of short helices, which are in the library, easily stitch into a long helix. This example generalizes to other protein tertiary motifs.

The methods, media, and systems provided by the disclosure are understood with reference to terms known in the art as well as the following terms.

A “tertiary motif” is a spatially local protein backbone arrangement consisting of one or more disjoint fragments. Tertiary motifs with a single disjoint segment are self-tertiary motifs. Tertiary motifs with two disjoint segments are pair tertiary motifs. Tertiary motifs with more than two disjoint segments can be named in a similar pattern. A tertiary motif can be considered a stand-in for the geometry it represents. For example, a short helical fragment can be a self-tertiary motif. Two disjoint helical fragments orientated, in a 3D orientation, with respect to each other in a certain way can be an exemplary pair tertiary motif. A person having ordinary skill in the art can recognize that the orientation of the disjoint helical fragments can be defined by two angles. A fragment of a beta-strand oriented in a specific way relative to a fragment of a short helix could also be a pair tertiary motif. These localized three-dimensional conformational models of a polypeptide include a sequence model that, for a given tertiary motif of n residues, for all n-mer amino-acid combinations, of which the n-mers may be either contiguous or made up of two or more non-overlapping segments, assigns a score that indicates the likelihood of the n-mer conforming to the given tertiary motif.

A “library of tertiary motifs” (e.g., a library) is a collection of two or more tertiary motifs (e.g., at least 100, 200 300, 400, 500, 600, 700, 800, 900, 1000, 2000, 3000, 4000, 5000, 10000, 15000, or 20000 tertiary motifs). One exemplary pre-existing library of tertiary motifs is described in “Tertiary Alphabet for the Observable Protein Structural Universe”, C. O. Mackenzie, J. Zhou and G. Grigoryan, Proceedings of the National Academy of Sciences, 113(47): E7438-E7447, 2016 (hereinafter “Mackenzie”).

A “probable tertiary motif,” (e.g., a probable self-tertiary motif or probable pair tertiary motif), is a set of tertiary motifs having a score assigned by a three-dimensional conformational model meeting a threshold for the subject protein, such as a fixed numerical scoring cutoff, a rank-order (e.g., top-n tertiary motifs), or a combination thereof. An appropriate value for the threshold depends on the implementation and specifics of the sequence model. In embodiments, a reference value cutoff (e.g., threshold) in employed in such a way as to balance false positive predictions (e.g., false positive predictions can include sequence windows that score better than the threshold, but do not, in fact, occupy the tertiary motif in question) and false negative predictions (e.g., sequence windows that score less well than the threshold, but do, in fact, occupy the tertiary motif in question). This balance can be identified using a training set of protein structures, where the tertiary motif identity of every possible sequence window is known. In some embodiments, the model employed estimates the minus log likelihood of observing the tertiary motif in question, conditioned upon the sequence (e.g., −log(p(tertiary motif sequence)). In certain more particular embodiments, a threshold of 3.2 (e.g., corresponding to a likelihood of ˜4%) is employed, although a person having ordinary skill in the art understands that other implementations can use other thresholds with different likelihoods.

A “rank-order” can be, for example, a percentile range, such as top 50, 40, 30, 20, 10, 9, 8, 7, 6, 5, 4, 3, 2, or 1%-ile). A person having ordinary skill in the art can recognize that a percentile range of “top n %” can be equivalent to (100−n) % to 100%. However, different expressions (e.g., inverse expressions) of data could lead to the range being 0%-n %.

A “configuration” is a vector of coordinates representing a polypeptide, and in some embodiments, represents each atom in a polypeptide (e.g. a subject protein).

Features of the methods, media, and systems described herein can include one or more of aspects of the following enumerated embodiments, which can be combined and interpolated and should not be viewed as narrow specific embodiments not amenable to combination or modulation, unless specifically provided. The examples of the instant disclosure provide non-limiting exemplification that can readily be adapted to the referenced embodiments.

Embodiment 1: Methods of predicting the structure of a subject protein comprising:

accepting the primary amino acid sequence of the subject protein;
accessing a library of tertiary motifs;
providing to a structural sampler a topology dataset comprising: a) a set of probable self-tertiary motifs from the library of tertiary motifs for the subject protein and b) a set of probable pair tertiary motifs from the library of tertiary motifs for the subject protein; and obtaining from the structural sampler a predicted structure representing a local minimum according to a scoring function incorporating a distance between a configuration and the probable tertiary motifs, wherein the structural sampler samples configurations of the subject protein according to the topology dataset, the structural sampler calculating the scoring function.

Embodiment 2: The methods of embodiment 1, wherein the distance is calculated as the root-mean squared deviation between a tertiary motif from the library and a corresponding fragment from the sampled configuration.

Embodiment 3: The methods of any one of the preceding embodiments, wherein the structural sampler samples configurations of the subject protein dynamically using Langevin dynamics, e.g., at a suitable pseudo thermodynamic temperature, e.g., about: 0.1, 0.5, 1.0, 5.0, 10.0, or 20.0 2.0. The pseudo thermodynamic temperature employed must be consistent with the magnitude and distribution of scores produced by the scoring function. If the thermodynamic temperature is beta, then 1/beta is a measure of a score difference that would be considered “significant” by the sampler. The higher the beta, the more the sampler will be exploring only the local space, paying attention to only conformations with low (“good”) scores. The lower the beta, the more we will be hopping around from local minimum to local minimum, and generally not descending to low (“good”) scoring sequences.

Embodiment 4: The methods of any one of the preceding embodiments, wherein the structural sampler samples configurations of the subject protein by Monte Carlo sampling.

Embodiment 5: The methods of any one of the preceding embodiments, wherein the scoring function is

$$E ( C ) = - {\sum }_{i} {w}_{i} \cdot {e}^{- β {r}_{i}^{2} ( C )} \qquad ( 1 )$$

Embodiment 6: The methods of any one of the preceding embodiments, wherein the structural sampler continuously optimizes chain coordinates to minimize the scoring function.

Embodiment 7: The methods of embodiment 5 or 6, wherein the scoring function is minimized by steepest descent minimization, e.g., by the method of Arfken, G. “The Method of Steepest Descents.” § 7.4 in Mathematical Methods for Physicists, 3rd ed. Orlando, Fla.: Academic Press, pp. 428-436, 1985.

Embodiment 8: The methods of embodiment 5 or 6, wherein the scoring function is minimized by conjugate gradients minimization, e.g., by the method of Straeter, T. A. “On the Extension of the Davidon-Broyden Class of Rank One, Quasi-Newton Minimization Methods to an Infinite Dimensional Hilbert Space with Applications to Optimal Control Problems”. NASA Technical Reports Server. NASA. hdl:2060/19710026200.

Embodiment 9: The methods of any one of the preceding embodiments, wherein the scoring function, in addition to the topology dataset, is provided one or more molecular mechanical features.

Embodiment 10: The methods of embodiment 9, wherein the one or more molecular mechanical features include bond, angle, and dihedral energies; van der Waals and Coulombic interaction energies; solvation energies; and combinations of the foregoing.

Embodiment 11: The methods of any one of the preceding embodiments, wherein the topology dataset further comprises a set of probable higher-order tertiary motifs, e.g., triplet tertiary motifs, quadruple tertiary motifs, pentuple tertiary motifs, or 1, 2, or all 3 of the foregoing.

Embodiment 12: The methods of any one of the preceding embodiments, wherein the set of probable self-tertiary motifs is determined by evaluating self-tertiary motifs in the library by each contiguous segment along the length of the subject protein according to the sequence model of the self-tertiary motif to provide a score that indicates the likelihood of the n-mer conforming to the tertiary motif, and identifying the set of probable self-tertiary motifs as those for which the score meets or exceeds a reference value. The appropriate value for the threshold depends on the specifics of the sequence model. In some embodiments, the reference value cutoff (threshold) in such a way as to balance false positive predictions (i.e., sequence windows that score better than the threshold, but do not, in fact, occupy the tertiary motif in question) and false negative predictions (i.e., sequence windows that score less well than the threshold, but do, in fact, occupy the tertiary motif in question). This balance can be identified using a training set of protein structures, where the tertiary motif identity of every possible sequence window is known. In some embodiments, the model employed estimates the minus log likelihood of observing the tertiary motif in question, conditioned upon the sequence (i.e., −log(p(tertiary motif|sequence)). In certain more particular embodiments, a threshold of 3.2 (corresponding to a likelihood of ˜4%) is employed, although the skilled artisan will appreciate that other implementations can use other thresholds with different likelihoods.

Embodiment 13: The methods of any one of the preceding embodiments, wherein the set of probable pair tertiary motifs is determined by evaluating pair tertiary motifs in the library by each non-overlapping pair of contiguous segments along the length of the subject protein according to the sequence model of the pair tertiary motif to provide a score that indicates the likelihood of the segments conforming to the tertiary motif, and identifying the set of probable pair tertiary motifs as those for which the score meets or exceeds a reference value. The considerations enumerated in embodiment 12 apply here mut. mut.

Embodiment 14: The methods of embodiment 12 or 13, wherein the reference value is a pre-determined numerical threshold or pre-determined rank-order (e.g., percentile, such as top 50, 40, 30, 20, 10, 9, 8, 7, 6, 5, 4, 3, 2, or 1%-ile).

Embodiment 15: The methods of any one of the preceding embodiments, wherein the library of tertiary motifs is sampled, e.g., up to about: 5, 10, 15, 20, 25, 30, 35, 40, 45, 50, 55, 60, 65, 70, 75, 80, 85, 90, or 95% to identify probable tertiary motifs, e.g., in the sample schemes, in certain embodiments, the tertiary motifs in the library are sampled according to their frequency in a reference database, such as PDB, that is, sampling first from the most frequently-occurring tertiary motifs to the least frequent tertiary motifs.

Embodiment 16: The methods of any one of the preceding embodiments, wherein the library of tertiary motifs is sampled exhaustively to identify probable tertiary motifs.

Embodiment 17: The methods of any one of the preceding embodiments, wherein the self-tertiary motifs are: 3, 4, 5, 6, 7, 8, 9, 10, 11, 12, 13, 14, or 15 residues, e.g., 3-10 residues or 4-6 residues.

Embodiment 18: The methods of any one of the preceding embodiments, wherein the pair tertiary motifs each have 2, 3, 4, 5, 6, or 7, e.g., 2-4 residues.

Embodiment 19: The methods of any one of the preceding embodiments, wherein the library of tertiary motifs was trained on a structural database comprising at least 500, 1000, 1500, 3000, 5000, 10000, 15000, 30000 unique structures, optionally wherein the structures have a resolution of less than about: 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, or 1.0 Angstroms, optionally wherein the structure is an X-ray crystallographic structure.

Embodiment 20: Wherein the structural database is the protein data bank (PDB).

Embodiment 21: The methods of any one of the preceding embodiments, wherein the self-tertiary motifs in the library have length n and are generated by clustering all contiguous n-mers in the library, e.g., by best-fit RMSD of backbone atoms or Euclidian distance map norm difference, e.g., by greedy clustering, k-means clustering, or hierarchical clustering.

Embodiment 22: The methods of any one of the preceding embodiments, wherein the pair tertiary motifs in the library have length n and are generated by identifying interacting residue pairs and generating a pair of n-mer tertiary motifs, e.g., wherein interacting residue pairs are identified distance between alpha carbon atoms (e.g., less than: 26, 24, 22, 20, 19, 18, 17, 16, 15, 14, 13, 12, 11, 10, 9, 8, 7, 6, 5, or 4 angstroms, e.g., less than 12 or 10 angstroms), distance between residue centroids (e.g., less than: 26, 24, 22, 20, 19, 18, 17, 16, 15, 14, 13, 12, 11, 10, 9, 8, 7, 6, 5, or 4 angstroms, e.g., less than 12 or 10 angstroms), contact degree-based definition (e.g., contact degree less than: 0.8, 0.7, 0.6, 0.5, 0.4, 0.3, 0.2, 0.1, 0.05, 0.025, 0.01, 0.005, 0.0025, 0.0001), or other residue orientation-depending geometric descriptors.

Embodiment 23: The methods of any one of the preceding embodiments, wherein the pair tertiary motifs in the library have length n and are generated by clustering all pairs of n-mer tertiary motifs in the library, e.g., by best-fit RMSD of backbone atoms or Euclidian distance map norm difference, e.g., by greedy clustering, k-means clustering, or hierarchical clustering.

Embodiment 24: The methods of any one of the preceding embodiments, wherein the component segments of pair tertiary motifs are both the same length.

Embodiment 25: The methods of any one of embodiments 1-23, wherein the component segments of pair tertiary motifs are different lengths.

Embodiment 26: The methods of any one of the preceding embodiments, wherein the sequence model of the tertiary motifs is generated by a Potts model of tertiary motifs in a cluster.

Embodiment 27: The methods of any one of the preceding embodiments, wherein the sequence model of the tertiary motifs is generated by a weak coupling framework of tertiary motifs in a cluster.

Embodiment 28: The methods of any one of the preceding embodiments, wherein the subject protein is a de novo protein, without a known homologue.

Embodiment 29: The methods of any one of the preceding embodiments, wherein the subject protein is less than 3000, 2000, 1000, 900, 800, 700, 600, 500, 400, 300, 200, 100, 90, 80, 70, 60, 40, 40, or 30 amino acids in length.

Embodiment 30: The methods of any one of the preceding embodiments, wherein the predicted structure exhibits a backbone RMSD less than about: 3.5, 3.4, 3.3, 3.2, 3.1, 3.0, 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7, 0.6, 0.5 Angstroms, relative to an experimentally-derived structure, e.g., an NMR structure, cryoEM, or X-ray crystal structure. In certain embodiments, RMSD can be calculated in other ways than backbone RMSD, such as over backbone and side chains or calculated relative to a subset of backbone or a subset of backbone and side chains.

Embodiment 31: A non-transient computer-readable medium comprising instructions that, upon execution by a microprocessor, causes the microprocessor to perform the methods of any one of the preceding embodiments.

Embodiment 32: A system comprising the non-transient computer-readable medium of embodiment 30 and a processor for executing the instructions, optionally wherein the system comprises one or more of a human end-user interface and a means for displaying the predicted structure.

Embodiment 33: A method of predicting the structure of a subject protein comprising providing the system of embodiment 31 with the primary amino acid sequence of the subject protein and obtaining the predicted structure.

Embodiment 34: The methods of any one of the preceding embodiments, wherein the steps are executed on a local server.

Embodiment 35: The methods of any one of the preceding embodiments, wherein one or more of the steps are executed on one or more remote servers.

### EXEMPLIFICATION

### Example 1: Methods

At the core of the approach is the concept of a tertiary motif, also referred to throughout as a “TERM,” which refers to a spatially local protein backbone arrangement having one or more disjoint fragments. The methodology employs a pre-built TERM library and a collection of motif geometries. The methodology applies the pre-built TERM library to guide the conformational sampling of a protein chain with a known sequence. Each motif in the TERM library has its own corresponding sequence model. Each model has a score indicating the compatibility between sequence and pre-generated library of tertiary motifs. The library can include the score, or the system can calculate the score. The structural sampler is based on the score of the motif, and can be implemented in a variety of ways as are known to a person having ordinary skill in the art.

The corresponding sequence, given any sequence equal to the length of the TERM, computes a score that indicates how likely the sequence is to conform to the TERM geometry. Given the models of each motif in the TERM library, and a sequence of interest S, it is then possible to pre-identify which TERMs are likely to be found within the structure taken by S, and how they are likely to align onto S. Finally, the pre-identified TERMs that are likely to be found within the structure S are processed by the structural sampling protocol to identify the optimal structure for S.

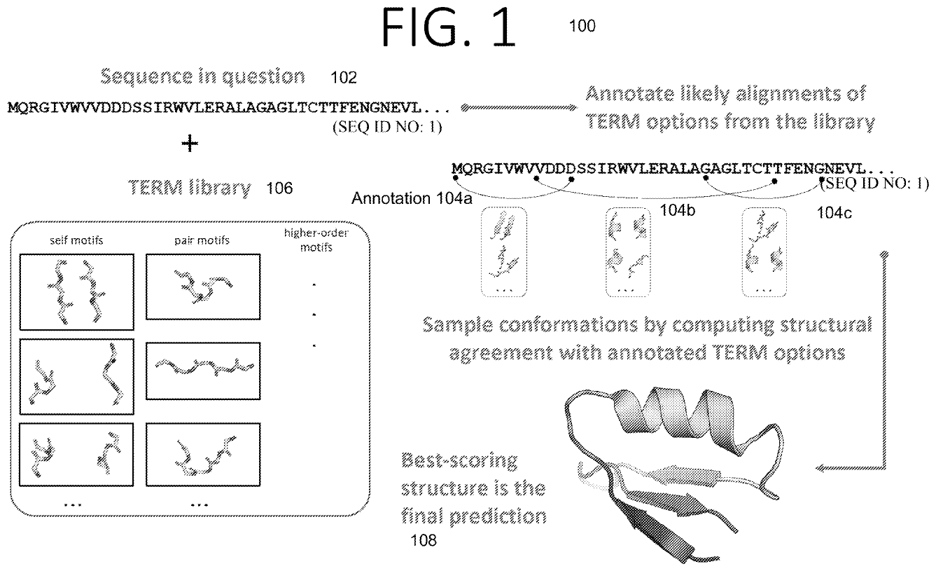

FIG. 1 is a diagram illustrating this process. A sequence in question 102, SEQ ID NO: 1, is analyzed in view of a TERM library 106 to generate annotations 104a-c that are alignments of the sequence in question 102 (SEQ ID NO: 1) annotated TERM options. The process samples conformations by computing structural agreement with annotated TERM options (e.g., calculating a score of each annotation 104a-c). The best scoring structure is outputted as the final prediction 108.

Building the TERM Library

Building a TERM library uses a structural database of proteins (e.g., the Protein Data Bank or PDB) and includes two steps: 1) identifying library TERMs as representatives of common geometries, and 2) building sequence models for library TERMs. In embodiments, the subject protein is less than 3000, 2000, 1000, 900, 800, 700, 600, 500, 400, 300, 200, 100, 90, 80, 70, 60, 40, 40, or 30 amino acids in length.

1) Identifying Library TERMs

To identify library TERMS, the method queries a structural database to identify commonly recurring geometries. In embodiments, the structural database has a particular size, and example embodiments has at least 1000, 1500, 3000, 5000, 10000, 15000, or 30000 unique structures. The library includes two classes of motifs: self-TERMs (e.g., TERMs made up of a single contiguous segment of residues) and pair TERMs (e.g., TERMs made up of two disjoint segments). In an embodiment, the approach can employ multi-segment TERMs beyond two segments (referred to as higher-order TERMs). In an embodiment, the lengths of each class of TERMs are fixed. For example, self-TERMs can be fixed at a particular number of residues long (e.g., 5) and pair TERMs can be fixed at another configuration of lengths (e.g., two segments that are each three residues long). However, in other embodiments TERMs can have multiple lengths per class in the library.

In an embodiment, clustering can identify commonly recurring geometries from a database. Each self-tertiary motif in the library can be considered in the context of each sequence window along the subject protein. A score is calculated each time to determine how good of a match the window is for the motif Given a local sequence window and a self-tertiary motif, the score indicates how good of a match the local sequence window and the self-tertiary motif are for each other. In other words, the score indicates the probability that the local sequence window conforms to that self-tertiary motif.

For example, for self-TERMs, first the method isolates all local 5-residue contiguous fragments and performs clustering using best-fit root mean square deviation (RMSD) of backbone atoms as the distance metric. In an embodiment, greedy clustering can be employed, but other clustering methods can also be used in other embodiments. For pair TERMs, the method first identifies all “interacting residues pairs” in the structural database. “Interacting residue pairs” are residue pairs that are positioned relative to each other in three-dimensional (3D) space in a way that can support a physical interaction. This can be defined in a variety of ways, such as (a) distance between alpha carbon atoms (e.g., less than: 26, 24, 22, 20, 19, 18, 17, 16, 15, 14, 13, 12, 11, 10, 9, 8, 7, 6, 5, or 4 angstroms), (b) distance between residue centroids (e.g., less than: 26, 24, 22, 20, 19, 18, 17, 16, 15, 14, 13, 12, 11, 10, 9, 8, 7, 6, 5, or 4 angstroms), (c) contact degree-based definition (e.g., use in the exemplified implementation) (e.g., contact degree less than: 0.8, 0.7, 0.6, 0.5, 0.4, 0.3, 0.2, 0.1, 0.05, 0.025, 0.01, 0.005, 0.0025, 0.0001), or (d) other residue orientation-depending geometric descriptors. Given a pair of interacting residues i−j, the method defines a pair TERM by combining residues {i−n, . . . , i+n} and residues {j−n, . . . , j+n} into a two-segment motif In an embodiment, n=1, but in other embodiments n=2 or other values are possible. Having defined a pair TERM for each interacting residue pair, the method performs clustering as described above (e.g., using best-fit RMSD as the distance metric) to arrive at representative TERM geometries. The method can perform a similar clustering procedure to obtain representative higher-order TERMs as well.

2) Building Sequence Models for Library TERMs

Each library TERM is a representative member of an entire cluster of structurally similar motifs, with each member of each cluster having a known sequence because each representative member arises from a structural database. The sequences of cluster members corresponding to a given TERM can therefore be used to construct a sequence model corresponding to the TERM geometry. This can be done in one of many ways. In a first embodiment, the method can build a Potts model using a maximum-likelihood approach, as described further in “Improved contact prediction in proteins: Using pseudolikelihoods to infer Potts models,” by Ekeberg et al., Physical Review E, 87(1) (hereinafter “Ekeberg”). In a second embodiment, the method can employ a weak coupling framework reported by “Direct-coupling analysis of residue coevolution captures native contacts across many protein families” by Morcos et al., Proceedings of the National Academy of Sciences December 2011, 108 (49) E1293-E1301; DOI:10.1073/pnas.1111471108 (hereinafter “Morcos”). A person having ordinary skill in the art can recognize that in other embodiments, other methods of building sequence models can be employed. Each TERM in the library has a corresponding model, by whatever suitable method, such that any given sequence of the right length can be scored as to the likelihood of the given sequence forming the geometry of each TERM. For example, if pair TERMs in the library are pairs of three-residue segments, then the method can determine, for sequence LKSTMS, which pair TERM is most likely to be found with amino acids LKS in one of its segments and TMS on the other.

In one implementation, the Potts model for each TERM tᵢ is built to approximate log(p(tᵢ|\vec{s})), where p(tᵢ|\vec{s}) is the probability of observing the geometry described by tᵢ given the sequence \vec{s}.

Annotating the Input Sequence

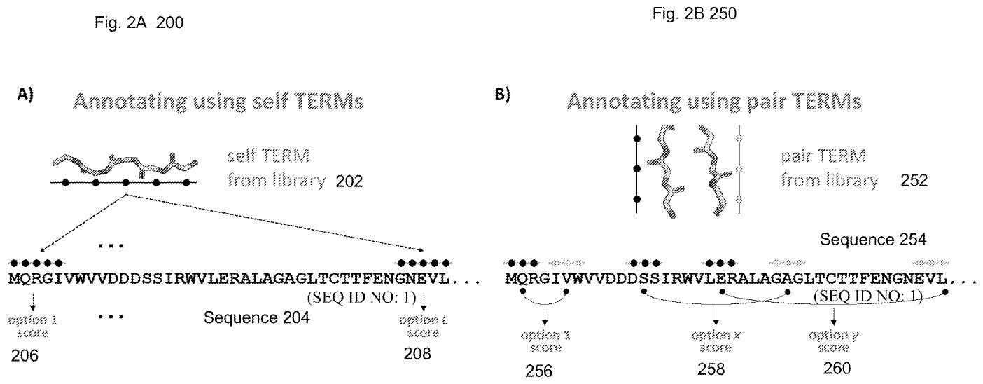

FIGS. 2A and 2B are diagrams 200 and 250 illustrating the annotation process on the example of a self-TERM 202 and pair TERM 252, respectively. In each case, the TERM is aligned onto the sequences 204 and 254 in all possible ways (e.g., addresses), with each address producing a score (e.g., score 206, 208, 256, 258, and 260) from the corresponding TERM model from the TERM library. The process is similar for higher-order TERMs, except that there are more possible addresses to consider.

Given a TERM library and an input sequence S of length L, the annotation process illustrated in FIGS. 2A-B includes scoring each TERM tᵢ in the library using the model associated with tᵢ in the context of each possible alignment it can have with S. For example, for a self-TERM of length l, the model scores each of the L−l+1 possible l-long contiguous sequence windows along S. Similarly, for a pair TERM, the model scores all possible unique non-overlapping alignment of the two segments onto contiguous sequence windows of S. For a higher-order TERM, the method scores all possible higher-order sequence window combinations. The method further can extend to cases where structure prediction involves multiple chains. In such cases, the method accounts for the fact that alignments where a single TERM segment spans multiple chains are not allowed.

The method scores alignment of a given TERM according to the TERM's model in conjunction with the sequence induced by the alignment. The method discards poorly scoring alignments and preserves well-scoring alignments by adding the well-scoring alignments to a list of geometry options for the alignment region. Thus, each geometry option includes: 1) an alignment region on S it pertains to (e.g., the address), 2) a TERM from the library, and 3) an associated score (e.g., the result of applying the TERM's sequence model onto the alignment region of S). The choice of which geometry options are preserved can be implemented in several ways: 1) by retaining only those options with scores above/below a pre-selected threshold or cutoff, 2) by preserving a given number of top-scoring options for each address (e.g., the top 20, top 50, or any other grouping of top scoring options), or 3) by a mixed approach, applying a cutoff together with a minimal number of options per address.

Together, the set of all geometry options at each address is referred to as the topology of the sequence S and information within it is used to perform structure prediction.

Scoring Function and Sampling

Given a pre-computed topology of S, the configuration of the protein chain is sampled according to a scoring function implied by the topology. A variety of scoring functions is possible, but a common ingredient is the RMSD between the TERM from a topology option and the current configuration of the chain at the address of the option. For example, for a self-TERM (of length l) from the i-th topology option having an address starting with residue k in the sequence, the method calculates the RMSD between the TERM and the chain fragment between residues k and k+l−1. Hereafter, this RMSD value is referred to as rᵢ(c), where subscript i designates that this RMSD corresponds to TERM from the i-th topology option and the value is a function of the current chain configuration C. Given this RMSD value, an exemplary simple scoring function (one used in the below examples) is:

$$E ( C ) = - {\sum }_{i} {w}_{i} \cdot {e}^{- β {r}_{i}^{2} ( C )} \qquad ( 1 )$$

where the sum is over all topology options, weights wᵢ determine the relative importance of the i-th option, and parameter β is a pseudo-temperature factor that controls the steepness of the potential as the RMSD to a given option's TERM changes. Weights wᵢ can be derived from the score of corresponding topology options, as the score indicates the likelihood that the sequence at the option's address takes on the configuration of the option's TERM. In other embodiments, the weights can all be fixed.

Equation (1) is differentiable, such that forces implied by this pseudo-energy can be calculated and the chain geometry can be sampled dynamically (e.g., using Langevin dynamics at a suitably chosen pseudo-temperature). Also, chain coordinates can be continuously optimized to minimize the score function (e.g., via steepest descent minimization or conjugate gradients minimization). Alternatively, the chain can be sampled with a cycle between discrete conformational moves and score evaluation (e.g., via Monte Carlo sampling). The sampled conformation with the lowest score constitutes the final predicted structure.

Sampling the library can include sampling a range of values of the library for their frequency to identify tertiary motifs in the sample schemes. For example, sampling can include sampling up to at least the most 5% frequent of the library. In other embodiments, sampling can include sampling up to at least 10, 15, 20, 25, 30, 35, 40, 45, 50, 55, 60, 65, 70, 75, 80, 85, 90, or 95% frequent of the library. In other words, sampling includes sampling first from the most frequently-occurring tertiary motifs to the least frequent tertiary motifs. In embodiments, the library can be PDB.

In embodiments, the scoring function can be optionally supplemented with additional components, such as “traditional” molecular-mechanics contributions (e.g., bond, angle, and dihedral energies; van der Waals and Coulombic interaction energies; solvation energies, etc.). However, in several examples that were tested, function in equation 1 was sufficient to predict the final structure well.

As demonstration of the techniques of the present disclosure, this methodology was applied to three examples. In two examples, the structure prediction task involves a single chain. In one example, the folding and complexation of two chains was considered. In each example, the process was the same as outlined in the Method section above, with the structural database extracted from the PDB to include all protein structures solved by X-ray crystallography to resolution better than 2.6 Å, pruned for redundancy at the level of 30% per-chain sequence identity. In other embodiments, the resolution can be at about: 3.0 2.9, 2.8, 2.7, 2.6, 2.5, 2.4, 2.3, 2.2, 2.1, 2.0, 1.9, 1.8, 1.7, 1.6, 1.5, 1.4, 1.3, 1.2, 1.1, 1.0, 0.9, 0.8, 0.7 angstroms, or fewer. Further, the TERM library was created with by greedy clustering using an RMSD cutoff of 0.7 Å for self-TERMs (5-residues long) and 1.0 Å for pair TERMs (two segments each 3-residues long). A Potts model was created for each cluster, from sequences of all cluster members, using a maximum-pseudolikelihood approach similar to the one described in Ekeberg 2013. The library contained 2,615 self-TERMs and 17,192 pair TERMs. In all cases below, the sampling approach to predict the final structure used Langevin Dynamics in conjunction with the scoring function in equation 1. In example embodiments, the Langevin Dynamics are employed at a suitable pseudo thermodynamic temperature, e.g., about: 0.1, 0.5, 1.0, 5.0, 10.0, or 20.0. The pseudo thermodynamic temperature employed should be consistent with the magnitude and distribution of scores produced by the scoring function. If the thermodynamic temperature is beta, then reciprocal of beta (e.g., 1/beta) is a measure of a score difference considered “significant” by the sampler. The higher the value of beta, the more the sampler explores only the local space, paying attention to only conformations with low (e.g., good/favorable) scores. The lower the value of beta, the more a model deviate around from local minimum to local minimum, and generally not descending to low (e.g., good/favorable) scoring sequences. The initial chain configuration was generated by choosing backbone phi and psi angles uniformly randomly and fixing the omega backbone angle to 180°.

### Example 2: A β3α Mini-Protein

SEQ ID NO: 2 is a mini-protein, with a β3α topology, that was designed de novo. See “Global Analysis of Protein Folding Using Massively Parallel Design, Synthesis, and Testing” by Rocklin et al. Science. 2017 Jul. 14; 357(6347):168-175 (hereinafter “Rocklin”). SEQ ID NO: 2 does not (by definition) have any sequence homologues in nature because it is a de-novo designed protein. Thus, a computational method cannot have inadvertently memorized the structure of this sequence by having seen structures of homologous sequences. Furthermore, the structure of the protein designated by SEQ ID NO: 2 was not among the set of structures from which the TERM library was built, as the structure was solved by NMR and not X-ray crystallography. For this reason, testing the instant approach on this de-novo protein represents a highly stringent test because the method should be able to generalize the process of protein sequence to structure mapping to recover the correct structure in this case.

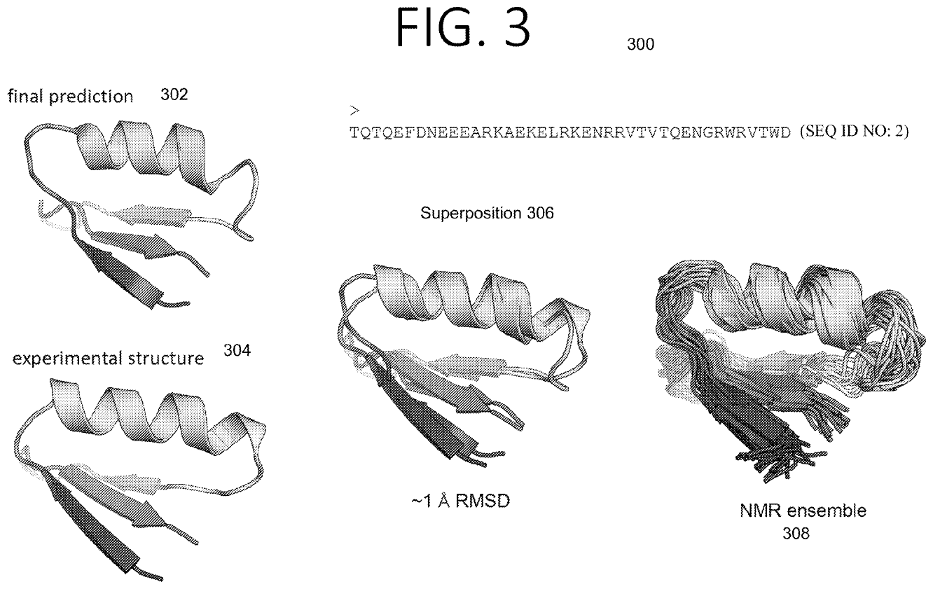

For this example, the full structure prediction calculation for protein designated by SEQ ID NO: 2 took on the order of several minutes on a laptop computer utilizing a single CPU core. FIG. 3 is a diagram 300 illustrating a resulting final predicted structure for this sequence (SEQ ID NO: 2), compared to the true experimental structure. The experimental structure can be an NMR structure or X-ray crystal structure. Notable, the prediction exhibits roughly a 1.0 Å backbone RMSD relative the top NMR model, and is well within the experimentally inferred NMR ensembled. In relation to FIG. 3, the left column illustrates a final predicted structure 302 and the selected best scoring/top member of the experimental NRM ensemble 304. Superposition 306 illustrates the superposition of the predicted structure and experimental structure 304. NMR ensemble 308 illustrates the superposition of the entire set of structures. FIG. 3 also illustrates the sequence of 5UP5.

### Example 3: An α3 Mini-Protein

As another test example, the same exact procedure is applied as above to the protein represented by SEQ ID NO: 3, a different de-novo protein. See Bhardwaj et al., “Accurate De Novo Design of Hyperstable Constrained Peptides,” Nature, 538(7625), 329-335 (2016) (hereinafter “Bhardwaj”). The same logic applies here as with the protein represented by SEQ ID NO: 3 above in relation to FIG. 3, as a de-novo designed protein, this sequence has no natural homologues, and due to being solved by NMR, the structure was not in the database from which the TERM library was built. So, once again, the methodology cannot “cheat” and instead must be able to generalize the correspondence between protein sequence and structure to recover the correct structure. As with the previous example, this calculation took several minutes on a single core processor.

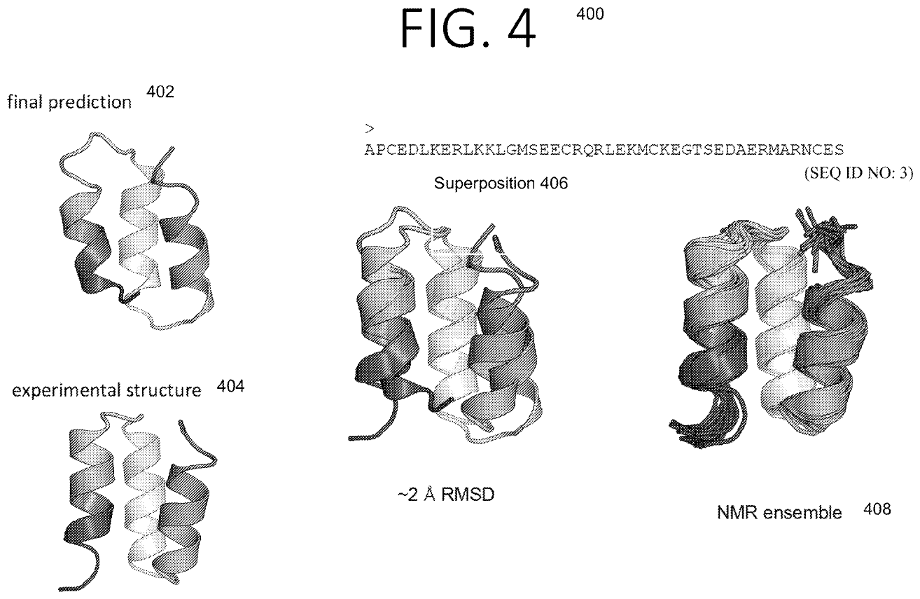

FIG. 4 is a diagram illustrating a final predicted structure compared to the experimental model. Once again, the agreement is very close (˜2 Å backbone RMSD between the top NMR model and the prediction), especially considering the breadth of the experimental NRM ensemble. In reference to FIG. 4, a final prediction 402 illustrates the final predicted structure and the top member 404 of the experimental NRM ensemble 408. A superposition 406 illustrates a superposition between the two structures 402 and 404. SEQ ID NO: 3 is illustrated at the top.

### Example 4: Dimeric Coiled Coil

As noted above, the approach of the present disclosure generalizes to cases of multi-chain structure prediction since nothing in the methodology assumes a single chain. Nevertheless, successfully testing the approach in a scenario having the additional challenge of having to differentiate between intra- and inter-chain interactions, the relative strengths of which are, in general, dependent on species concentrations, further validated the approach. To test the approach in this regime, the case of a dimeric coiled coil from a yeast protein represented by SEQ ID NO: 4, a classic workhorse system for studying coiled coils and dimeric association principles, was considered. To perform this experiment, two chains of random conformations with coincident centroids initialize the system. In this way, the sampling encourages identification of interactions between and within chains, while no information was given about the way the chains may associate or in what orientation. The simulation time in this case roughly 30 minutes on a single core.

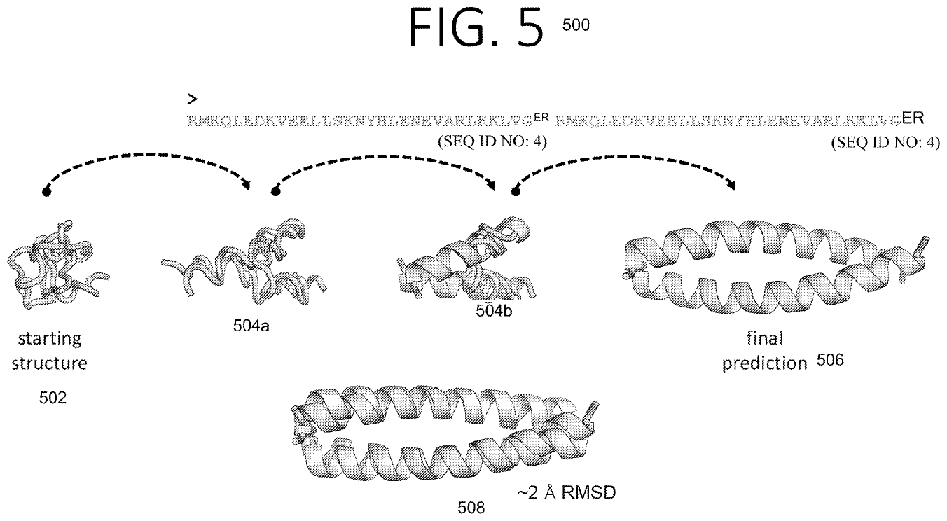

FIG. 5 is a diagram 500 illustrating a resulting folded predicted structure 506, a starting (random) structure 502, as well as several intermediary structures/snapshots 504a-b along the sampling trajectory. A superposition 508 illustrates the final prediction 506 in blue and green, representing the two chains of the protein represented by SEQ ID NO: 4, and the true structure in white. Once again, the correct structure was recovered at the end of sampling, exhibiting ˜2 Å backbone RMSD with the true experimental structure. The orientation of the two helical chains was predicted correctly (e.g., that the helical chains are in parallel), which is noteworthy because the free energy gap between association states of alternative orientation in coiled coils is known to be generally small.

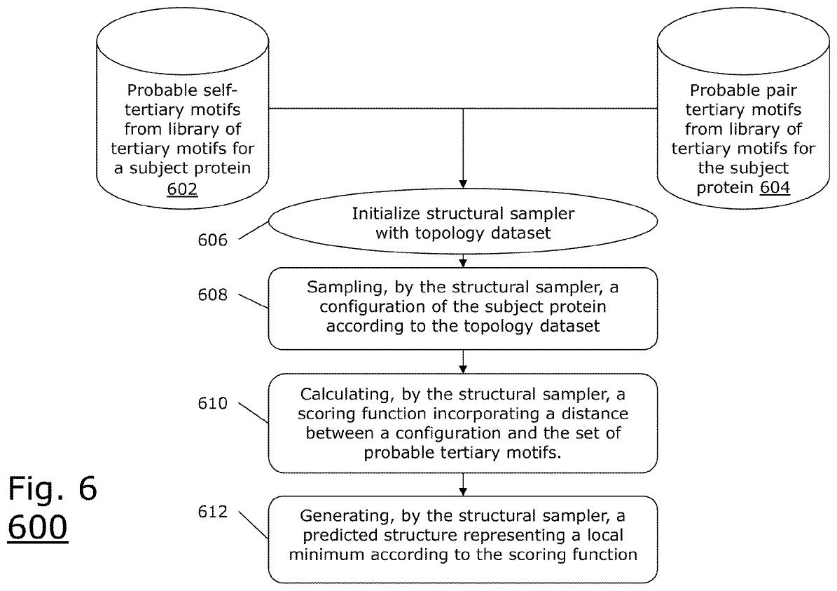

FIG. 6 is a flow diagram 600 illustrating an example embodiment of a method employed by the present disclosure. The method initializes a structural sampler with a topology dataset that includes probable self-tertiary motifs from library of tertiary motifs for a subject protein 602 and probable pair tertiary motifs from library of tertiary motifs for the subject protein 604 (606). Then, the method samples, by the structural sampler, a configuration of the subject protein according to the topology dataset (608). The method then calculates, by the structural sampler, a scoring function incorporating a distance between a configuration and the set of probable tertiary motifs (610). In other words, the method proposes different configurations of the subject protein, and then compares the TERMS formed by the configuration versus the set of probable pre-computed TERMS from the library. When the comparison is favorable, that increases the score, and when the comparisons are not favorable, they decrease the score. A person having ordinary skill in the art can recognize that multiple samplings and respective scorings can be performed. The method then generates, by the structural sampler, a predicted structure representing a local minimum according to the scoring function (612).

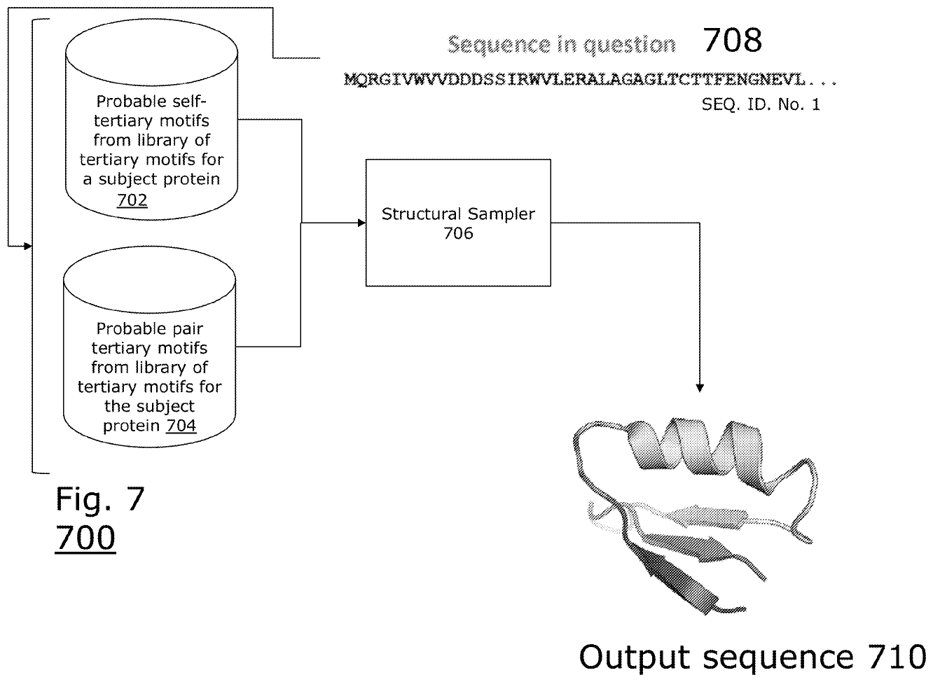

FIG. 7 is a block diagram 700 illustrating an example embodiment of the present disclosure. A structural sampler 706 is implemented by a processor and a memory. In embodiments, the structural sampler 706 can be implemented by one or more serves (e.g., in the cloud). The structural sampler 706 is initialized by probable self-tertiary motifs from library of tertiary motifs for a subject protein 702 and probable pair tertiary motifs from library of tertiary motifs for the subject protein 704 loaded from respective databases or data sources. The sample in question 708 is used to load the probable self-tertiary motifs and probably pair tertiary motifs from their respective libraries. The structural sampler 706 annotates the structure in question 708, as also illustrated in FIG. 1, where the annotations represent likely alignments of tertiary motifs (TERM) options from the library. The structural sampler 706 computes structural agreement for each of the likely alignments of the TERM options by calculating a score for each alignment. The structural sampler 706 then outputs the highest scoring alignment as the output sequence 710. A person having ordinary skill in the art can recognize that the output sequence 710 can be subsequently manufactured.

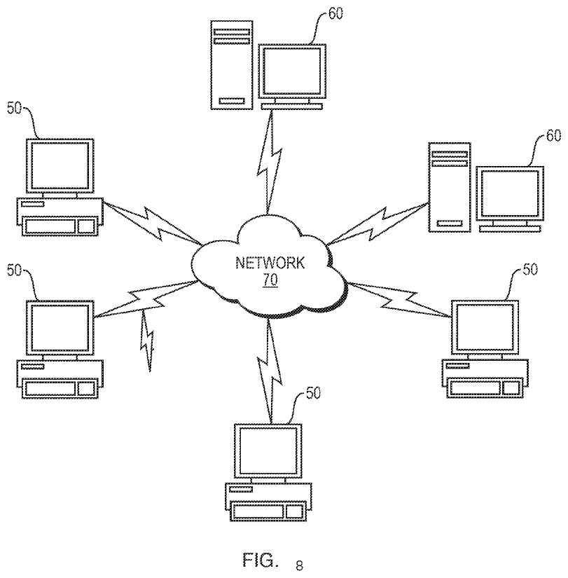

FIG. 8 illustrates a computer network or similar digital processing environment in which embodiments of the present invention may be implemented.

Client computer(s)/devices 50 and server computer(s) 60 provide processing, storage, and input/output devices executing application programs and the like. The client computer(s)/devices 50 can also be linked through communications network 70 to other computing devices, including other client devices/processes 50 and server computer(s) 60. The communications network 70 can be part of a remote access network, a global network (e.g., the Internet), a worldwide collection of computers, local area or wide area networks, and gateways that currently use respective protocols (TCP/IP, Bluetooth®, etc.) to communicate with one another. Other electronic device/computer network architectures are suitable.

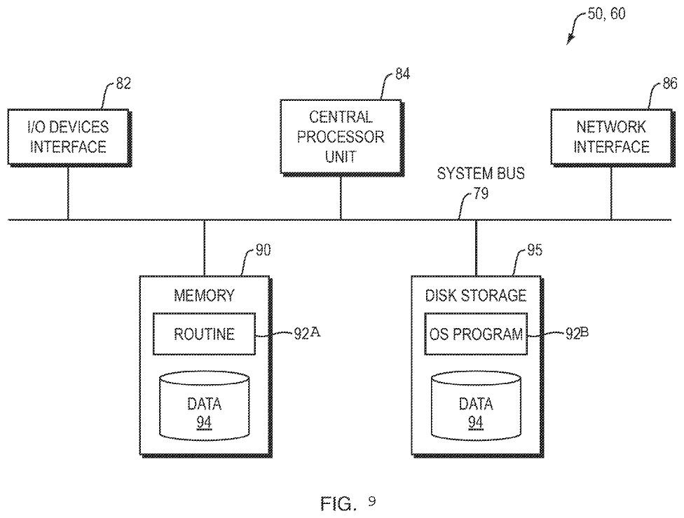

FIG. 9 is a diagram of an example internal structure of a computer (e.g., client processor/device 50 or server computers 60) in the computer system of FIG. 8. Each computer 50, 60 contains a system bus 79, where a bus is a set of hardware lines used for data transfer among the components of a computer or processing system. The system bus 79 is essentially a shared conduit that connects different elements of a computer system (e.g., processor, disk storage, memory, input/output ports, network ports, etc.) that enables the transfer of information between the elements. Attached to the system bus 79 is an I/O device interface 82 for connecting various input and output devices (e.g., keyboard, mouse, displays, printers, speakers, etc.) to the computer 50, 60. A network interface 86 allows the computer to connect to various other devices attached to a network (e.g., network 70 of FIG. 5). Memory 90 provides volatile storage for computer software instructions 92 and data 94 used to implement an embodiment of the present invention (e.g., structural sampler code detailed above). Disk storage 95 provides non-volatile storage for computer software instructions 92 and data 94 used to implement an embodiment of the present invention. A central processor unit 84 is also attached to the system bus 79 and provides for the execution of computer instructions.

In one embodiment, the processor routines 92 and data 94 are a computer program product (generally referenced 92), including a non-transitory computer-readable medium (e.g., a removable storage medium such as one or more DVD-ROM's, CD-ROM's, diskettes, tapes, etc.) that provides at least a portion of the software instructions for the invention system. The computer program product 92 can be installed by any suitable software installation procedure, as is well known in the art. In another embodiment, at least a portion of the software instructions may also be downloaded over a cable communication and/or wireless connection. In other embodiments, the invention programs are a computer program propagated signal product embodied on a propagated signal on a propagation medium (e.g., a radio wave, an infrared wave, a laser wave, a sound wave, or an electrical wave propagated over a global network such as the Internet, or other network(s)). Such carrier medium or signals may be employed to provide at least a portion of the software instructions for the present invention routines/program 92.

A person having ordinary skill in the art can understood that for all numerical bounds describing some parameter in this application, such as “about,” “at least,” “less than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, etcetera.

For all patents, applications, or other reference cited herein, such as non-patent literature and reference sequence information, it should be understood that they are incorporated by reference in their entirety for all purposes as well as for the proposition that is recited. Where any conflict exists between a document incorporated by reference and the present application, this application will control. All information associated with reference gene sequences disclosed in this application, such as GeneIDs or accession numbers (typically referencing NCBI accession numbers), including, for example, genomic loci, genomic sequences, functional annotations, allelic variants, and reference mRNA (including, e.g., exon boundaries or response elements) and protein sequences (such as conserved domain structures), as well as chemical references (e.g., PubChem compound, PubChem substance, or PubChem Bioassay entries, including the annotations therein, such as structures and assays, et cetera), are hereby incorporated by reference in their entirety.

Headings used in this application are for convenience only and do not affect the interpretation of this application.

Preferred features of each of the aspects provided by the disclosure are applicable to all of the other aspects of the disclosure mutatis mutandis and, without limitation, are exemplified by the dependent claims and also encompass combinations and permutations of individual features (e.g., elements, including numerical ranges and exemplary embodiments) of particular embodiments and aspects of the disclosure, including the working examples. For example, particular experimental parameters exemplified in the working examples can be adapted for use in the claimed disclosure piecemeal without departing from the disclosure. For example, for materials that are disclosed, while specific reference of each of the various individual and collective combinations and permutations of these compounds may not be explicitly disclosed, each is specifically contemplated and described herein. Thus, if a class of elements A, B, and C are disclosed as well as a class of elements D, E, and F and an example of a combination of elements A-D is disclosed, then, even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-groups of A-E, B-F, and C-E are specifically contemplated and should be considered disclosed from disclosure of A, B, and C; D, E, and F; and the example combination A-D. This concept applies to all aspects of this application, including elements of a composition of matter and steps of method of making or using the compositions.

The forgoing aspects of the disclosure, as recognized by the person having ordinary skill in the art following the teachings of the specification, can be claimed in any combination or permutation to the extent that they are novel and non-obvious over the prior art—thus, to the extent an element is described in one or more references known to the person having ordinary skill in the art, they may be excluded from the claimed disclosure by, inter alia, a negative proviso or disclaimer of the feature or combination of features.

It should be understood that the example embodiments described above may be implemented in many different ways. In some instances, the various methods and machines described herein may be implemented by a physical, virtual, or hybrid general purpose computer, or a computer network environment.

Embodiments or aspects thereof may be implemented in the form of hardware, firmware, or software. If implemented in software, the software may be stored on any non-transient computer-readable medium that is configured to enable a processor to load the software or subsets of instructions thereof. The processor then executes the instructions and is configured to operate or cause an apparatus to operate in a manner as described herein.

Further, firmware, software, routines, or instructions may be described herein as performing certain actions and/or functions of the data processors. However, it should be appreciated that such descriptions contained herein are merely for convenience and that such actions, in fact, result from computer devices, processors, controllers, or other devices executing the firmware, software, routines, instructions, etc.

It should also be understood that the schematics may include more or fewer elements, be arranged differently, or be represented differently. But it should further be understood that certain implementation may dictate that the schematic be implemented in a particular way.

The described computer-readable implementations may be implemented in software, hardware, or a combination of hardware and software. Examples of hardware include computing or processing systems, such as personal computers, servers, laptops, mainframes, and micro-processors. In addition, one of ordinary skill in the art will appreciate that the records and fields shown in the figures may have additional or fewer fields, and may arrange fields differently than the figures illustrate. Any of the computer-readable implementations provided by the disclosure may, optionally, further comprise a step of providing a visual output to a user, such as a visual representation of, for example, the predicted structure of a subject protein, e.g., to an end user, such a user accessing a system or non-transient computer-readable medium described herein.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of predicting the structure of a subject protein comprising:
initializing a structural sampler with a topology dataset comprising: a) a set of probable self-tertiary motifs from a library of tertiary motifs for a subject protein and b) a set of probable pair tertiary motifs from the library of tertiary motifs for the subject protein;
sampling, by the structural sampler, at least one configuration of the subject protein according to the topology dataset;
calculating, by the structural sampler, a scoring function incorporating a distance between a configuration and the set of probable tertiary motifs; and
generating, by the structural sampler, a predicted structure representing a local minimum according to the scoring function.

2. The method of claim 1, further comprising calculating the distance as the root-mean squared deviation between a tertiary motif from the library and a fragment from the at least one configuration sampled corresponding to a structure of the tertiary motif.

3. The method of claim 1, wherein sampling, by the structural sampler, includes sampling the at least one configuration of the subject protein dynamically using at least one of Langevin dynamics and Monte Carlo sampling.

4. (canceled)

5. The method of claim 1, wherein the scoring function is
$$E ( C ) = - {\sum }_{i} {w}_{i} \cdot {e}^{- β {r}_{i}^{2} ( C )} \qquad ( 1 )$$

6. The method of claim 1, further comprising:
continuously optimizing chain coordinates of the structural sampler to minimize the scoring function.

7. The method of claim 5, wherein the scoring function is minimized by at least one of steepest descent minimization or conjugate gradients minimization.

8. The method of claim 1, wherein the scoring function, in addition to the topology dataset, utilizes one or more molecular mechanical features.

9. The method of claim 8, wherein the one or more molecular mechanical features include one or more of:
bond, angle, and dihedral energies;
van der Waals and Coulombic interaction energies; and
solvation energies.

10. The method of claim 1, wherein the topology dataset further comprises a set of at least one of triplet tertiary motifs, quadruple tertiary motifs, pentuple tertiary motifs, and probable higher-order tertiary motifs.

11. The method of claim 1, further comprising:
determining the set of probable self-tertiary motifs by evaluating the self-tertiary motifs in the library by comparing each contiguous segment along a length of the subject protein according to a sequence model of the self-tertiary motif,
calculating a score that indicates a probability of the n-mer conforming to the tertiary motif, or providing a score that indicates a probability of the segments conforming to the tertiary motif, and
identifying the set of probable self-tertiary motifs as those for which the score meets or exceeds a reference value.

12. (canceled)

13. The method of claim 11, wherein the reference value is a pre-determined numerical threshold or pre-determined rank-order.

14. The method of claim 1, wherein sampling includes at least one of:
sampling the library of tertiary motifs according to their frequency in a reference database to identify probable tertiary motifs; and
sampling the library of tertiary motifs exhaustively to identify probably tertiary motifs.

15-21. (canceled)

22. The method of claim 1, wherein the self-tertiary motifs in the library have a length n, and further comprising:
generating the self-tertiary motifs by clustering all contiguous n-mers in the library.

23. The method of claim 22, wherein clustering all contiguous n-mers in the library is performed by at least one of best-fit RMSD of backbone atoms or Euclidian distance map norm difference.

24. The method of claim 23, wherein Euclidian distance map norm difference is performed by at least one of greedy clustering, k-means clustering, or hierarchical clustering.

25. The method of claim 1, wherein the pair tertiary motifs in the library have a length n and further comprising:
generating the pair tertiary motifs by identifying interacting residue pairs having a distance between alpha carbon atoms and generating a pair of n-mer tertiary motifs having at least one of interacting residue pairs, distance between residue centroids, contact degree-based definition, and other residue orientation-depending geometric descriptors.

26. The method of claim 25, wherein at least one of:
interacting residue pairs have a distance between alpha carbon atoms of less than 26 angstroms;
distance between residue centroids is less than 25 angstroms; and
contract degree-based definition is a contact degree less than 0.8.

27. The method of claim 1, wherein the pair tertiary motifs in the library have a length n, and further comprising:
generating the pair tertiary motifs by clustering all pairs of n-mer tertiary motifs in the library.

28. The method of claim 27, wherein clustering all pairs of n-mer tertiary motifs in the library is performed by at least one of best-fit RMSD of backbone atoms and Euclidian distance map norm difference.

29. The method of claim 28, wherein the Euclidian distance map norm difference is performed by at least one of greedy clustering, k-means clustering, and hierarchical clustering.

30. The method of claim 1, wherein the component segments of pair tertiary motifs are both the same length.

31. The method of claim 1, wherein the component segments of pair tertiary motifs are different lengths.

32. The method of claim 1, further comprising generating the sequence model of the tertiary motifs by employing at least one of a Potts model of tertiary motifs in a cluster and a weak coupling framework of tertiary motifs in a cluster.

33. (canceled)

34. The method of claim 1, wherein the subject protein is at least one of a de novo protein without a known homologue and less than 3000 amino acids in length.

35. (canceled)

36. The method of claim 1, wherein the predicted structure exhibits a backbone RMSD less than 3.5 Angstroms, relative to an experimentally-derived structure.

37. A non-transient computer-readable medium comprising instructions that, upon execution by a microprocessor, causes the microprocessor to perform the method of claim 1.

38. A system comprising the non-transient computer-readable medium of claim 36 and a processor for executing the instructions, optionally wherein the system comprises one or more of a human end-user interface and a means for displaying the predicted structure.

39. A method of predicting the structure of a subject protein comprising providing the system of claim 31 with a primary amino acid sequence of the subject protein and obtaining the predicted structure.

40-41. (canceled)
